SEQUENCE LISTING (116 sequences)

SEQ ID NO: 1 - protein, 5 aa, Artificial Sequence
  Synthetic Peptide
FVFLM

SEQ ID NO: 2 - protein, 5 aa, Artificial Sequence
  Synthetic Peptide
FVFAM

SEQ ID NO: 3 - protein, 5 aa, Artificial Sequence
  Synthetic Peptide
FVALM

SEQ ID NO: 4 - protein, 5 aa, Artificial Sequence
  Synthetic Peptide
FVFLA

SEQ ID NO: 5 - protein, 5 aa, Artificial Sequence
  Synthetic Peptide
FLVFI

SEQ ID NO: 6 - protein, 5 aa, Artificial Sequence
  Synthetic Peptide
FLMII

SEQ ID NO: 7 - protein, 5 aa, Artificial Sequence
  Synthetic Peptide
FLFVL

SEQ ID NO: 8 - protein, 5 aa, Artificial Sequence
  Synthetic Peptide
FLFVV

SEQ ID NO: 9 - protein, 5 aa, Artificial Sequence
  Synthetic Peptide
FLFLI

SEQ ID NO: 10 - protein, 5 aa, Artificial Sequence
  Synthetic Peptide
FLFFI

SEQ ID NO: 11 - protein, 5 aa, Artificial Sequence
  Synthetic Peptide
FLMFI

SEQ ID NO: 12 - protein, 5 aa, Artificial Sequence
  Synthetic Peptide
FMLLI

SEQ ID NO: 13 - protein, 5 aa, Artificial Sequence
  Synthetic Peptide
FIIMI

SEQ ID NO: 14 - protein, 5 aa, Artificial Sequence
  Synthetic Peptide
FLFCI

SEQ ID NO: 15 - protein, 5 aa, Artificial Sequence
  Synthetic Peptide
FLFAV

SEQ ID NO: 16 - protein, 5 aa, Artificial Sequence
  Synthetic Peptide
FVYLI

SEQ ID NO: 17 - protein, 5 aa, Artificial Sequence
  Synthetic Peptide
FAFLM

SEQ ID NO: 18 - protein, 5 aa, Artificial Sequence
  Synthetic Peptide
AVFLM

SEQ ID NO: 19 - protein, 10 aa, Artificial Sequence
  Synthetic Peptide
MPSSVSWGIL

SEQ ID NO: 20 - protein, 10 aa, Artificial Sequence
  Synthetic Peptide
LAGLCCLVPV

SEQ ID NO: 21 - protein, 10 aa, Artificial Sequence
  Synthetic Peptide
SLAEDPQGDA

SEQ ID NO: 22 - protein, 10 aa, Artificial Sequence
  Synthetic Peptide
AQKTDTSHHD

SEQ ID NO: 23 - protein, 10 aa, Artificial Sequence
  Synthetic Peptide
QDHPTFNKIT

SEQ ID NO: 24 - protein, 10 aa, Artificial Sequence
  Synthetic Peptide
PNLAEFAFSL

SEQ ID NO: 25 - protein, 10 aa, Artificial Sequence
  Synthetic Peptide
YRQLAHQSNS

SEQ ID NO: 26 - protein, 10 aa, Artificial Sequence
  Synthetic Peptide
TNIFFSPVSI

SEQ ID NO: 27 - protein, 10 aa, Artificial Sequence
  Synthetic Peptide
ATAFAMLSLG

SEQ ID NO: 28 - protein, 10 aa, Artificial Sequence
  Synthetic Peptide
TKADTHDEIL

SEQ ID NO: 29 - protein, 10 aa, Artificial Sequence
  Synthetic Peptide
EGLNFNLTEI

SEQ ID NO: 30 - protein, 10 aa, Artificial Sequence
  Synthetic Peptide
PEAQIHEGFQ

SEQ ID NO: 31 - protein, 10 aa, Artificial Sequence
  Synthetic Peptide
ELLRTLNQPD

SEQ ID NO: 32 - protein, 10 aa, Artificial Sequence
  Synthetic Peptide
SQLQLTTGNG

SEQ ID NO: 33 - protein, 10 aa, Artificial Sequence
  Synthetic Peptide
LFLSEGLKLV

SEQ ID NO: 34 - protein, 10 aa, Artificial Sequence
  Synthetic Peptide
DKFLEDVKKL

SEQ ID NO: 35 - protein, 10 aa, Artificial Sequence
  Synthetic Peptide
YHSEAFTVNF

SEQ ID NO: 36 - protein, 10 aa, Artificial Sequence
  Synthetic Peptide
GDHEEAKKQI

SEQ ID NO: 37 - protein, 10 aa, Artificial Sequence
  Synthetic Peptide
NDYVEKGTQG

SEQ ID NO: 38 - protein, 10 aa, Artificial Sequence
  Synthetic Peptide
KIVDLVKELD

SEQ ID NO: 39 - protein, 10 aa, Artificial Sequence
  Synthetic Peptide
RDTVFALVNY

SEQ ID NO: 40 - protein, 10 aa, Artificial Sequence
  Synthetic Peptide
IFFKGKWERP

SEQ ID NO: 41 - protein, 10 aa, Artificial Sequence
  Synthetic Peptide
FEVKDTEDED

SEQ ID NO: 42 - protein, 10 aa, Artificial Sequence
  Synthetic Peptide
FHVDQVTTVK

SEQ ID NO: 43 - protein, 10 aa, Artificial Sequence
  Synthetic Peptide
VPMMKRLGMF

SEQ ID NO: 44 - protein, 10 aa, Artificial Sequence
  Synthetic Peptide
NIQHCKKLSS

SEQ ID NO: 45 - protein, 10 aa, Artificial Sequence
  Synthetic Peptide
WVLLMKYLGN

SEQ ID NO: 46 - protein, 10 aa, Artificial Sequence
  Synthetic Peptide
ATAIFFLPDE

SEQ ID NO: 47 - protein, 10 aa, Artificial Sequence
  Synthetic Peptide
GKLQHLENEL

SEQ ID NO: 48 - protein, 10 aa, Artificial Sequence
  Synthetic Peptide
THDIITKFLE

SEQ ID NO: 49 - protein, 10 aa, Artificial Sequence
  Synthetic Peptide
NEDRRSASLH

SEQ ID NO: 50 - protein, 10 aa, Artificial Sequence
  Synthetic Peptide
LPKLSITGTY

SEQ ID NO: 51 - protein, 10 aa, Artificial Sequence
  Synthetic Peptide
DLKSVLGQLG

SEQ ID NO: 52 - protein, 10 aa, Artificial Sequence
  Synthetic Peptide
ITKVFSNGAD

SEQ ID NO: 53 - protein, 10 aa, Artificial Sequence
  Synthetic Peptide
LSGVTEEAPL

SEQ ID NO: 54 - protein, 10 aa, Artificial Sequence
  Synthetic Peptide
KLSKAVHKAV

SEQ ID NO: 55 - protein, 10 aa, Artificial Sequence
  Synthetic Peptide
LTIDEKGTEA

SEQ ID NO: 56 - protein, 10 aa, Artificial Sequence
  Synthetic Peptide
AGAMFLEAIP

SEQ ID NO: 57 - protein, 10 aa, Artificial Sequence
  Synthetic Peptide
MSIPPEVKFN

SEQ ID NO: 58 - protein, 10 aa, Artificial Sequence
  Synthetic Peptide
KPFVFLMIEQ

SEQ ID NO: 59 - protein, 10 aa, Artificial Sequence
  Synthetic Peptide
NTKSPLFMGK

SEQ ID NO: 60 - protein, 7 aa, Artificial Sequence
  Synthetic Peptide
VVNPTQK

SEQ ID NO: 61 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 62 - protein, 394 aa, Artificial Sequence
  Synthetic Polypeptide
  MISC_FEATURE (356)..(357): Xaa = any amino acid
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDHEEAKKQINDYVEKGTQGKIVDLVKELDRDTVFALVNYIFFKGKWERPFEVKDTEDEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLERXXRSIPPEVKFNKPFVFLMIEQNTKSPLFMGKVVNPTQK

SEQ ID NO: 63 - protein, 417 aa, Homo sapiens
MPSSVSWGILLAGLCCLVPVSLAEDPQGDAAQKTDTSHHDQDHPTFNKITPNLAEFAFSLYRQLAHQSNSTNIFFSPVSIATAFANLSLGTKADTHDEILEGLNFNLTEIPEAQIHEGFQELLRTLNQPDSQLQLTTGNGLFLSEGLKLVDKFLEDVKKLYHSEAFTVNFGDHEEAKKQINDYVEKGTQGKIVDLVKELDRDTVFALVNYIFFKGKWERPFEVKDTEDEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 64 - DNA, 699 bp, Artificial Sequence
  Synthetic Polynucleotide
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 65 - protein, 232 aa, Artificial Sequence
  Synthetic Polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - DNA, 1257 bp, Homo sapiens
atgccgtcttctgtctcgtggggcatcctcctgctggcaggcctgtgctgcctggtccctgtctccctggctgaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaatga

SEQ ID NO: 67 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 68 - DNA, 1185 bp, Homo sapiens
gaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaatga

SEQ ID NO: 69 - protein, 394 aa, Homo sapiens; identical to SEQ ID NO: 61

SEQ ID NO: 70 - DNA, 1959 bp, Artificial Sequence
  Synthetic Polynucleotide AAT-FC
atgccgtcttctgtctcgtggggcatcctcctgctggcaggcctgtgctgcctggtccctgtctccctggctgaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaaacgcgtgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 71 - protein, 652 aa, Artificial Sequence
  Synthetic Polypeptide AAT-FC
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKTREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 1953 bp, Artificial Sequence
  Synthetic Polynucleotide AAT-FC
atgccgtcttctgtctcgtggggcatcctcctgctggcaggcctgtgctgcctggtccctgtctccctggctgaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 73 - protein, 650 aa, Artificial Sequence
  Synthetic Polypeptide AAT-FC
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - DNA, 1881 bp, Artificial Sequence
  Synthetic Polynucleotide AAT-FC
gaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 75 - protein, 626 aa, Artificial Sequence
  Synthetic Polypeptide AAT-FC
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - DNA, 1257 bp, Homo sapiens
atgccgtcttctgtctcgtggggcatcctcctgctggcaggcctgtgctgcctggtccctgtctccctggctgaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaatga

SEQ ID NO: 77 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQATTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 78 - DNA, 1185 bp, Homo sapiens
gaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaatga

SEQ ID NO: 79 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQATTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 80 - DNA, 1959 bp, Artificial Sequence
  Synthetic Polynucleotide AAT-FC
atgccgtcttctgtctcgtggggcatcctcctgctggcaggcctgtgctgcctggtccctgtctccctggctgaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaaacgcgtgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 81 - protein, 652 aa, Artificial Sequence
  Synthetic Polypeptide AAT-FC
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQATTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKTREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - DNA, 1953 bp, Artificial Sequence
  Synthetic Polynucleotide AAT-FC
atgccgtcttctgtctcgtggggcatcctcctgctggcaggcctgtgctgcctggtccctgtctccctggctgaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 83 - protein, 650 aa, Artificial Sequence
  Synthetic Polypeptide AAT-FC
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQATTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - DNA, 1881 bp, Artificial Sequence
  Synthetic Polynucleotide AAT-FC
gaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 85 - protein, 626 aa, Artificial Sequence
  Synthetic Polypeptide AAT-FC
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQATTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - DNA, 1886 bp, Artificial Sequence
  Synthetic Polynucleotide AAT-FC
gaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaaacgcgtgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 87 - protein, 628 aa, Artificial Sequence
  Synthetic Polypeptide AAT-FC
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKTREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - DNA, 1886 bp, Artificial Sequence
  Synthetic Polynucleotide AAT-FC
gaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaaacgcgtgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 89 - protein, 628 aa, Artificial Sequence [sequence omitted]

SEQ ID NO: 90 - protein, 10 aa, Artificial Sequence
  Synthetic Peptide
GITKVFSNGA

SEQ ID NO: 91 - protein, 10 aa, Artificial Sequence
  Synthetic Peptide
DLSGVTEEAP

SEQ ID NO: 92 - protein, 10 aa, Artificial Sequence
  Synthetic Peptide
LKLSKAVHKA

SEQ ID NO: 93 - protein, 10 aa, Artificial Sequence
  Synthetic Peptide
VLTIDEKGTE

SEQ ID NO: 94 - protein, 10 aa, Artificial Sequence
  Synthetic Peptide
AAGAMFLEAI

SEQ ID NO: 95 - protein, 10 aa, Artificial Sequence
  Synthetic Peptide
PMSIPPEVKF

SEQ ID NO: 96 - protein, 10 aa, Artificial Sequence
  Synthetic Peptide
NKPFVFLMIE

SEQ ID NO: 97 - protein, 10 aa, Artificial Sequence
  Synthetic Peptide
QNTKSPLFMG

SEQ ID NO: 98 - protein, 8 aa, Artificial Sequence
  Synthetic Peptide
KVVNPTQK

SEQ ID NO: 99 - protein, 22 aa, Artificial Sequence
  Synthetic Polypeptide
LEAIPMSIPPEVKFNKPFVFLM

SEQ ID NO: 100 - protein, 20 aa, Artificial Sequence
  Synthetic Polypeptide
LEAIPMSIPPEVKFNKPFVF

SEQ ID NO: 101 - protein, 80 aa, Artificial Sequence
  Synthetic Polypeptide
GADLSGVTEEAPLKLSKAVHKAVLTIDEKGTEAAGAMFLEAIPMSIPPEVKFNKPFVFLMIEQNTKSPLFMGKVVNPTQK

SEQ ID NO: 102 - protein, 36 aa, Artificial Sequence
  Synthetic Polypeptide
SIPPEVKFNKPFVFLMIEQNTKSPLFMGKVVNPTQK

SEQ ID NO: 103 - DNA, 62 bp, Artificial Sequence
  Synthetic Polypeptide
tttagaggccatacccatgtctatcccccccgaggtcaagttcaacaaacccctttgtcttt

SEQ ID NO: 104 - DNA, 62 bp, Artificial Sequence
  Synthetic Polypeptide
tttagaggccatatgcatgtctatcccccccgaggtcaagttcaacaaacccctttgtcttt

SEQ ID NO: 105 - DNA, 1977 bp, Artificial Sequence
  Synthetic Polynucleotide AAT-FC
gaattcgccaccatgccgtcttctgtctcgtggggcatcctcctgctggcaggcctgtgctgcctggtccctgtctccctggctgaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaaacgcgtgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaggatct

SEQ ID NO: 106 - DNA, 1950 bp, Artificial Sequence
  Synthetic Polypeptide AAT-FC
gaattcgccaccatgccgtcttctgtctcgtggggcatcctcctgctggcaggcctgtgctgcctggtccctgtctccctggctgaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaaacgcgtacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaggatct

SEQ ID NO: 107 - protein, 643 aa, Artificial Sequence
  Synthetic Polypeptide AAT-FC
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQATTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKTRTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - DNA, 1962 bp, Artificial Sequence
  Synthetic Polynucleotide AAT-FC
gaattcgccaccatgccgtcttctgtctcgtggggcatcctcctgctggcaggcctgtgctgcctggtccctgtctccctggctgaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaaacgcgtcgcaaatgttgtgtcgagtgcccaccgtgcccagcaccacctgtggcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaggatct

SEQ ID NO: 109 - protein, 647 aa, Artificial Sequence
  Synthetic Polypeptide AAT-FC
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQATTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKTRRKCCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - DNA, 1995 bp, Artificial Sequence
  Synthetic Polynucleotide AAT-FC
gaattcgccaccatgccgtcttctgtctcgtggggcatcctcctgctggcaggcctgtgctgcctggtccctgtctccctggctgaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaaacgcgtccatgcccacggtgcccagagcccaaatcttgtgacacacctcccccgtgcccaaggtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaggatct

SEQ ID NO: 111 - protein, 658 aa, Artificial Sequence [sequence omitted]

SEQ ID NO: 112 - DNA, 1965 bp, Artificial Sequence
  Synthetic Polynucleotide AAT-FC
gaattcgccaccatgccgtcttctgtctcgtggggcatcctcctgctggcaggcctgtgctgcctggtccctgtctccctggctgaggatccccagggagatgctgcccagaagacagatacatcccaccacgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaaacgcgttccaaatatggtcccccatgcccatcatgcccagcacctgagttcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaggatct

SEQ ID NO: 113 - protein, 648 aa, Artificial Sequence
  Synthetic Polypeptide AAT-FC
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQATTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKTRSKYGPPCPSCPAPEFLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 634 aa, Artificial Sequence
  Synthetic Polypeptide AAT-FC
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQATTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKTRPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 632 aa, Artificial Sequence
  Synthetic Polypeptide AAT-FC
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQATTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKTRGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 632 aa, Artificial Sequence; identical to SEQ ID NO: 115
  Synthetic Polypeptide AAT-FC

(19) United States
(12) Patent Application Publication
(10) Pub. No.: US 2019/0008935 A1
(43) Pub. Date: Jan. 10, 2019

(54) METHODS AND USES FOR ALPHA-1 ANTITRYPSIN OR RECOMBINANT FORMS THEREOF, ON STEROID-REFRACTORY GRAFT VERSUS HOST DISEASE INVOLVING GASTROINTESTINAL COMPLICATIONS

(71) Applicants: THE REGENTS OF THE UNIVERSITY OF COLORADO, A BODO CORPORATE, DENVER, CO (US); FRED HUTCHINSON CANCER RESEARCH CENTER, SEATTLE, WA (US)

(72) Inventors: CHARLES DINARELLO, DENVER, CO (US); MARIO Q. MARCONDES, MILLBRAE, CA (US); H. JOACHIM DEEG, SEATTLE, WA (US)

(21) Appl. No.: 16/067,076

(22) PCT Filed: Dec. 30, 2016
(86) PCT No.: PCT/US2016/069563

§ 371 Date: Jun. 28, 2018

Related U.S. Application Data
(60) Provisional application No. 62/336,488, filed on May 13, 2016.
(60) Provisional application No. 62/274,107, filed on Dec. 31, 2015.

(51) Int. Cl. A61K 38/57; A61P 37/06; A61K 9/00; A61K 45/06

(52) U.S. Cl. CPC A61K 38/57; A61P 37/06; A61K 45/06; A61K 9/0019; A61K 9/0053; A61K 9/0073

(57) ABSTRACT

The application provides methods of treating a subject suffering from advanced-stage GvHD with α1-antitrypsin (AAT), any carboxy terminal peptide derivatives thereof, or any recombinant versions thereof. In certain embodiments, the subject is a glucocorticoid-refractory patient having GvHD with gut involvement. In other embodiments, donor subjects of a transplant are treated with AAT, any carboxy terminal peptide derivatives thereof, or any recombinant versions thereof, to reduce graft rejection or GvHD in a recipient subject.

This PCT application claims the benefit under 35 USC § 119(e) of provisional U.S. application No. 62/274,107, filed on Dec. 31, 2015 and provisional U.S. application No. 62/336,488, filed on May 13, 2016, each of which are incorporated herein by reference in their entirety for all purposes.

### FIELD

Embodiments described herein generally relate to methods for diagnosing a subject having steroid-refractory GVHD and treating the subject with alpha1-antitrypsin (α1-antitrypsin, AAT) and carboxyterminal peptide derivatives thereof, and/or immunomodulators or anti-inflammatory agents with activity similar to that of AAT. Certain embodiments herein concern regimens for treating a subject having advanced grade GvHD with gut involvement with AAT and optionally, with follow-on treatments of AAT after initial administration of AAT to the subject.

### SEQUENCE LISTING

The instant application contains a Sequence Listing which has been submitted electronically in ASCII format, and is hereby incoroporated by reference in its entirety. The ASCII copy, created on Dec. 30, 2016, is name 507751108 SEQLIST.txt, and is 168,978 bytes in size.

### BACKGROUND

### Serine Proteases

Serine proteases serve an important role in human physiology by mediating the activation of vital functions. In addition to their normal physiological function, serine proteases have been implicated in a number of pathological conditions in humans. Serine proteases are characterized by a catalytic triad consisting of aspartic acid, histidine and serine at the active site.

Typical plasma concentration of sertine protease, α1-antitrypsin (ATT) ranges from 1.3 to 3.5 mg/ml. It diffuses into tissue spaces and forms a 1:1 complex with target proteases, principally neutrophil elastase. Other enzymes such as trypsin, chymotrypsin, cathepsin G, plasmin, thrombin, tissue kallikrein, and factor Xa can also serve as substrates. The enzyme/inhibitor complex is then removed from circulation by binding to serpin-enzyme complex (SEC) receptor and catabolized by the liver and spleen. ATT appears to represent an important part of the defense mechanism against activity by serine proteases.

AAT is one of few naturally occurring mammalian serine protease inhibitors currently approved for the clinical therapy of protease imbalance. Therapeutic α1-antitrypsin has been commercially available since the mid 1980's and is prepared by various purification methods. Prolastin®, Glassia®, Aralast®, Zemaira®, amongst others, are trademarks for partially and/or purified AAT currently on the market.

### Graft Versus Host Disease (GvHD)

Graft-versus-host disease (GvHD) is a medical complication following the receipt of transplanted cells, tissue or organs from a genetically different person or donor. GvHD can also be associated with transfusion (e.g. blood transfusion) or other conditions. GvHD is commonly associated with stem cell transplant (bone marrow transplant), but the term also applies to other forms of transplantation. Immune cells (white blood cells) in the donated tissue (the graft) recognize the recipient (the host) as foreign (nonself). The transplanted immune cells then attack the host's body cells. GvHD can also occur after a blood transfusion if the blood products used have not been irradiated or treated with an approved pathogen reduction system. Transplant rejection occurs when the host rejects the graft, GvHD occurs when the graft rejects the host.

Bone marrow transplantation is a unique kind of transplant where immune cells from a donor are transferred into a recipient, thereby conferring the donor immune system into the recipient. The bone marrow is capable of generating an immune response against the host. Rigorous immunosuppressive and antimicrobial treatments can be required to block adverse consequences of GvHD. Considerable progress has been achieved with allogeneic hematopoietic cell transplantation (HCT) which can be attributed from bone marrow or hematopoietic cells obtained from blood or other means. However, GVHD remains a problem, developing in an acute form in 30-70% of patients despite prophylaxis with immunosuppressive agents. Therefore, a need exists for safer and more effective inhibitors of the adverse effects by the graft.

Because of some of the difficulties and inadequacies of conventional therapy for treating transplantation complications and associated side-effects, new therapeutic modalities are needed.

### SUMMARY

Embodiments described herein relate to methods for diagnosing a subject having steroid-refractory GVHD and treating the subject with alpha1-antitrypsin (α1-antitrypsin, AAT) and carboxyterminal peptide derivatives thereof, and/or immunomodulators or anti-inflammatory agents with activity similar to that of AAT. Certain embodiments herein concern regimens for treating a subject having advanced grade GvHD with gut involvement using a pharmaceutically acceptable composition having AAT and optionally, using follow-on treatments of AAT to about 30 days to 60 days after initial administration of AAT to the subject.

Certain embodiments concern methods for treating GvHD in a subject including identifying a subject having GvHD; identifying the subject having GvHD that is steroid-refractory acute GvHD; identifying the subject having steroid refractory acute GvHD of grade III or grade IV GvHD having stage 3 or 4 gut involvement; and administering a pharmaceutically acceptable formulation of a composition including alpha-1 antityrpsin (AAT), a recombinant of AAT thereof or an RCL mutant of AAT thereof (e.g. where the reactive center loop of AAT has one or more amino acid substitutions) or a carboxyterminal fragment thereof. In accordance with these embodiments, a subject can be treated with AAT or carboxyterminal fragments thereof in a pharmaceutically acceptable composition over a 5 to 60 day or longer, 5 to 30 day or 9 to 21 day or 10 to 15 day administration period depending on need wherein the composition is administered to the subject on day 1 of the administration period at a higher dose (e.g. concentration) than administered in one or more follow-on doses, thereby treating steroid refractory GvHD in the subject. In addition, methods can further include, evaluating the gut involvement by upper and lower gastro-intestinal evaluation prior to, during and after AAT treatment and documenting gut involvement in the subject and treating the subject based on improvement of gut symptoms. In accordance with these emdobiments, treatment of these GvHD subjects can occur over a single day to 60-day period or longer (e.g. one year) as required or where positive results are demonstrated in the subject for treating GvHD or gut involvement of the subject for example treatment with AAT for 1 year or more. In accordance with these embodiments, a dose of a composition can range from 1.0 mg/kg to 150.0 mg/kgAAT per dose administered to the subject.

In certain embodiments, a dose on day 1 administered to a subject can be about 1.5 to 10 times more concentrated than a follow-on dose. In accordance with these embodiments, the dose administered to the subject on day 1 can be from 3.0 mg/kg to 150 mg/kg. Further, follow-on dose(s) can be from 1.0 mg/kg to 100 mg/kg. In certain embodiments, the dose administered to the subject on day 1 can be about 90 mg/kg, while follow on dose can be 30 mg/kg to 60 mg/kg. In yet other embodiments, multiple doses can be administered to the subject on day 1 of the treatment. In certain embodiments, follow-on doses can be administered to a subject every day over the administration period. In other embodiments, the follow-on doses can be administered to a subject every other day over the administration period or other regimen as determined by a qualified health provider.

Certain embodiments herein concern methods for treating GvHD in a subject including identifying a subject having GvHD; identifying the subject having GvHD that has steroid-refractory GvHD having grade III or grade IV GvHD with stage 4 gut involvement; administering a pharmaceutically acceptable formulation of a composition including alpha-1 antityrpsin (AAT) or recombinant full-length AAT linked to an immunoglobulin Fc (e.g. IgG1, IgG2, IgG3, IgG4 or IgGD) or other suitable agent (e.g. GAG or other suitable fusion molecule) to the subject having GvHD with gut involvement and a pharmaceutically acceptable excipient and treating the subject.

Compositions disclosed herein can be administered immediately after diagnosis of a grade III or IV GvHD in a subject or some time later. In addition, compositions disclosed herein can further include one or more anti-transplant rejection agent, anti-inflammatory agent, immunosuppressive agent, immunomodulatory agent, anti-microbial agent, or a combination thereof.

Embodiments of the present disclosure provide for methods for ameliorating symptoms or signs experienced by a subject having graft versus host disease (GVHD), with significant gut involvement. In one example, methods disclosed herein can be used to treat a subject undergoing transplantation or other condition that leads to GvHD (e.g. acute GvHD) as provided herein.

Certain embodiments concern treating a subject with compositions disclosed herein in order to reduce or prevent late-stage GvHD-related fatalities. In accordance with these embodiments, the subject can be a subject having steroid refractory GvHD with stage 3 or 4 gut involvement wherein steroids no longer inhibit the subject's immune system. Compositions disclosed herein can be used to reduce gut involvement and/or prevent fatalities by prolonging the life of the subject. In accordance with these embodiments, administration of a pharmaceutically acceptable composition including AAT and/or carboxyterminal derived peptides of AAT or recombinant AAT thereof can be used to reduce gut complications and symptoms as well as reducing GvHD-related complications and death of the subject.

It is contemplated herein that AAT can include naturally occurring AAT harvested from human or other mammalian plasma and/or commercially available AAT formulations such as Aralast®, Zemaira®, Glassia®, Prolastin® and ProlastinC®. Therapeutically effective amounts of AAT can include those doses administered to AAT deficient patients or in a range of about 1.0 to about 150 mg/kg in a single or multiple dose regimen. It is contemplated herein that AAT or derivative thereof can be administered to a subject in need thereof and blood can be drawn from the subject in order to assess the level of AAT in the subject. In addition, once the level of AAT is determined, a health professional may administer more or less AAT to the subject depending on need and in order to maintain a predetermined blood concentration of AAT (e.g. 2.0 to 4.0 mg/mL).

In certain embodiments of the present disclosure, compositions disclosed herein can be combined with other anti-inflammatory compound or immunomodulatory drug. In accordance with these embodiments, anti-inflammatory compound or immunomodulatory drugs can include but are not limited to, one or more of interferon, interferon derivatives including betaseron, beta-interferon, prostane derivatives including iloprost, cicaprost; glucocorticoids including cortisol, prednisolone, methyl-prednisolone, dexamethasone; immunsuppressives including cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives including ACTH and analogs thereof; soluble TNF-receptors; TNF-αntibodies; soluble receptors of interleukins, other cytokines, T-cell-proteins; antibodies against receptors of interleukins, other cytokines, T-cell-proteins; and calcipotriols; Celcept®, mycophenolate mofetil, and analogues thereof taken either alone or in combination.

In some embodiments, AAT polypeptides contemplated for use in the compositions and methods of the present disclosure can include any and all of those specific AAT polypeptides represented in the Sequence Listing or other known AAT polypeptide. Any combination of consecutive amino acids depicting a portion of AAT or AAT-like activity can be used.

In one aspect, the pharmaceutical compositions of the present disclosure are administered orally, systemically, via an implant, intravenously, topically, intrathecally, intratracheally, intracranially, subcutaneously, intravaginally, intraventricularly, intranasally such as inhalation, mixed with grafts by flushing of organ or suspension of cells, or any combination thereof.

As such, those skilled in the art will appreciate that the conception, upon which this disclosure is based, can readily be used as a basis for designing other methods for carrying out the several features and advantages described herein.

### BRIEF DESCRIPTION OF THE DRAWINGS

The following drawings form part of the present specification and are included to further demonstrate certain embodiments of the present disclosure. The embodiments may be better understood by reference to one or more of these drawings in combination with the detailed description of specific embodiments presented herein.

FIGS. 1A and 1B represents exemplary photographic illustrations of A) a patient's gut demonstrating steroid-refractory acute GvHD before administration of a composition of embodiments disclosed herein; and B) a patient's gut demonstrating steroid-refractory acute GvHD after administration of a composition of embodiments disclosed herein.

FIGS. 2A to 2C represents graphic illustrations of levels of AAT treatment and concentration in a subject contemplated herein where A) represents plasma AAT concentrations of subjects treated with AAT; B) represents stool AAT concentration in subjects before and during AAT therapy and C) represents calculated AAT clearance on the basis of A) and B) in the subject.

FIG. 3 represents an exemplary graph of plasma TIM3 levels in steroid refractory GvHD subjects over the duration of AAT treatment.

FIG. 4 represents an exemplary graph of Treg levels in peripheral blood of steroid refractory GvHD subjects undergoing AAT treatment compared to control subjects not having steroid refractory GvHD over the course of AAT treatment in the treated GvHD subjects.

### DESCRIPTION OF ILLUSTRATIVE EMBODIMENTS

### Definitions

Terms that are not otherwise defined herein are used in accordance with their plain and ordinary meaning.

As used herein, “a” or “an” can mean one or more than one of an item.

As used herein “analog of alpha-1-antitrypsin” can mean a compound having alpha-1-antitrypsin-like activity such that the analog behaves the same or similarly to AAT. In one embodiment, an analog of alpha-1-antitrypsin can be a functional derivative of alpha-1-antitrypsin. In another embodiment, an analog of alpha-1-antitrypsin can be a compound capable of significantly reducing serine protease activity or inflammation and/or an immune response and/or reduces gut involvement in a steroid refractory GvHD subject.

As used herein “immunomodulatory drugs or agents”, can mean, e.g., agents which act on the immune system, directly or indirectly, e.g., by stimulating or suppressing an adverse cellular activity of a cell in the immune system, e.g., T-cells, B-cells, macrophages, or antigen presenting cells (APC, dendritic cells), or by acting upon components outside the immune system which, in turn, stimulate, suppress, or modulate the immune system, e.g. cytokines, e.g., hormones, receptor agonists or antagonists, and neurotransmitters; immunomodulators can be, e.g., immunosuppressants or immunostimulants.

It is to be understood that the terminology and phraseology and definitions employed herein are for the purpose of description and included as embodiments and should not be regarded as limiting.

### DETAILED DESCRIPTION

In the following sections, various exemplary compositions and methods are described in order to detail the various embodiments. It will be obvious to one skilled in the art that practicing the various embodiments does not require the employment of all or even some of the specific details outlined herein, but rather that concentrations, dose regimen, time of administration and other specific details may be modified through routine experimentation. In some embodiments, understood methods, regimens or components of one of skill in the art have not been included in the description.

Embodiments disclosed herein provide for methods for treating a subject having complications of GvHD or advanced-stage GvHD. In accordance with these embodiments, a subject may be treated with a composition capable of significantly reducing these GvHD-related complications or reducing them to nearly undetectable levels. In certain embodiments, methods disclosed herein include treating a subject having GvHD or advanced-stage GvHD with a composition comprising AAT or AAT carboxyterminus peptide or RCL AAT mutant or recombinant AAT molecule thereof to for example, to reduce GvHD-related complications in gut stage 3 or 4 GvHD or to increase survival thereof.

Some embodiments relate to methods for diagnosing a subject with steroid-refractory GVHD and treating the subject with α1-antitrypsin (AAT) or carboxyterminal AAT peptide derivatives thereof.

Certain embodiments concern methods for treating GvHD in a subject including identifying a subject having steroid refractory GvHD; identifying the subject having GvHD that has steroid-refractory GvHD having grade III or grade IV GvHD with stage 3 or 4 gut involvement; administering a pharmaceutically acceptable formulation of a composition including AAT, a recombinant thereof or a RCL mutant thereof or a carboxyterminal fragment thereof to the subject over a 1 to 30-day, or 5 to 25 day, or 9 to 21 day period of administration wherein the composition can be administered to the subject on day 1 of the administration period. In certain embodiments, the initial (e.g. day 1) administration is at a higher concentration than one or more follow-on doses.

In some embodiments, follow-on doses can be administered every day over the course of the administration period following the day 1 administration. In other embodiments, follow-on doses can be administered every other day over the course of the administration period following the initial administration of the compostion. For example, with a 9-day administration period, a high dose of the composition can be administered on day 1, with follow-on doses being administered on days 3, 5, 7, and 9 in an every other day administration regimen. In certain embodiments, an administration period can last 15 days. In accordance with these embodiments, an administration period can be 15 days, with follow on doses being administered every other day (e.g., days 3, 5, 7, 9, 11, 13, and 15) following administration on day 1. Alternatively, a subject can be treated every day over the course of a 15-day regimen. Other regimens can include a less frequent treatment regimen and a single follow-on dose is also contemplated after the initial treatment is administered to the subject.

In certain embodiments the dose administered to the subject on day 1 of the administration can be 1.5 to 10 times or 1.5 to 5.0 times higher AAT or AAT peptide concentration than the one or more follow-on doses. In some embodiments, the dose administered to the subject on day one of treatment can have a concentration of AAT or AAT peptide concentration from 3.0 mg/kg to 150 mg/kg. Follow-on doses can range from 1.0 mg/kg to about 100 mg/kg or 1.0 mg/kg to about 75 mg/kg or from 1.0 mg/kg to about 40 mg/kg etc. In certain embodiments, the concentration of AAT or carboxyterminal peptide fragment; or fusion polypeptide thereof administered to the subject on day 1 is about 90 mg/kg; or about 9 mg/kg respectively, with follow-on doses of about 30 mg/kg to about 60 mg/kg; or about 3.0 mg/kg to about 6.0 mg/kg respectively.

In addition, methods can further include, evaluating GvHD-related gut involvement by upper and lower gastro-intestinal evaluation of the subject prior to AAT treatment and documenting gut involvement in the subject. In other embodiments, treatment of these steroid refractory GvHD subjects can occur over a 1 to 60-day period or longer as required or where positive results are demonstrated in the subject for treating GvHD or gut involvement of the subject such as for 1 year or more. In accordance with these methods, a dose of a composition can range from 1.0 mg/kg to 150.0 mg/kg. In certain embodiments, a dose on day 1 can be about 1.5 to 10 times more concentrated than a follow-on dose.

Certain embodiments concern methods for treating GvHD in a subject including identifying a subject having GvHD that has steroid-refractory GvHD having grade III or grade IV GvHD with stage 4 gut involvement; administering a pharmaceutically acceptable formulation of a composition of alpha-1 antityrpsin (AAT) or recombinant full-length AAT linked to an immunoglobulin Fc or other fusion polypeptide or recombinant thereof to the subject having GvHD with gut involvement in order to treat GvHD or GvHD-related symptoms or signs in the subject. Compositions disclosed herein can be administered immediately after diagnosis of a grade III or IV GvHD in a subject or some time later. In addition, the composition administered to the affected subject may further include one or more anti-transplant rejection agent, anti-inflammatory agent, immunosuppressive agent, immunomodulatory agent, anti-microbial agent, or a combination thereof.

Certain embodiments provide for methods for ameliorating symptoms or signs experienced by a subject having advanced stage GVHD with gut involvement. In some embodiments, the subject can present with significant gut involvement which can be assessed throughout treatment as appropriate. In one example, methods disclosed herein can be used to treat a subject undergoing organ, tissue or cellular (non-organ) transplantation leading to acute GvHD as provided herein.

Certain embodiments disclosed herein concern treating a subject with AAT compositions in order to reduce or prevent late-stage GvHD-related fatalities or prolong survival of the subject. In other embodiments, administration of a composition including AAT and/or carboxyterminal derived peptides of AAT or recombinant thereof can be used to reduce gut complications and symptoms associated with late-stage acute GvHD in a subject.

It is contemplated herein that AAT can include naturally occurring AAT harvested from human or other mammalian plasma and/or commercially available formulations such as Aralast®, Zemaira®, Glassia® and Prolastin® and ProlastinC® or other plasma-derived AAT formulation. Therapeutically effective doses of AAT can be administered to AAT deficient patients or patients with a normal AAT level in a concentration range of about 1.0 mg/kg to about 150 mg/kg as single or multiple dose regimens. It is contemplated herein that AAT or derivative thereof can be administered to a subject in need thereof and blood can be drawn from the subject in order to assess the level of AAT in the subject. In addition, once the level of AAT is determined, a health professional may administer more or less AAT to the subject depending on need.

In certain embodiments, the subject is a human subject. In some embodiments, the human subject is any age meeting the GvHD grade and stage criteria. In yet other embodiments, the subject is a middle-aged subject having stage 3 or 4 gut involved GvHD. In certain embodiments, the human subject is 25 years or older.

In certain embodiments, the anti-inflammatory compound or immunomodulatory drug can include but is not limited to one or more of interferon, interferon derivatives including betaseron, beta-interferon, prostane derivatives including iloprost, cicaprost; glucocorticoids including cortisol, prednisolone, methyl-prednisolone, dexamethasone; immunsuppressives including cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives including ACTH and analogs thereof; soluble receptors of interleukins, other cytokines, T-cell-proteins; antibodies against receptors of interleukins, other cytokines, T-cell-proteins; and calcipotriols; Celcept®, mycophenolate mofetil, and analogues thereof taken either alone or in combination.

In another aspect, the present disclosure provides for a method of ameliorating or reducing a symptom or sign associated with advanced stage GvHD in a subject in need of said amelioration or reduction. In accordance with this embodiment, a pharmaceutically effective amount of AAT or carboxyterminal peptide fragment thereof or fusion polypeptide of AAT, wherein the composition is capable of reduce or eliminate late stage GvHD or symptom thereof.

Certain embodiments concern methods for inhibiting progression of late-stage GvHD in a subject by administering a pharmaceutically acceptable formulation of a composition including alpha-1 antityrpsin (AAT), a recombinant thereof or a RCL mutant thereof or a carboxyterminal fragment thereof to the subject.

In certain embodiments, during transplantation events, an organ, tissue or cell donor is selected to match (e.g., HLA matching) the subject scheduled to receive the transplantation or implantation. In other embodiments, the donor and subject are mis-matched. In accordance with these embodiments, compositions disclosed herein can be used to pre-treat an organ, tissue or cell donor subject with a composition described herein whether the donor is a match or mismatched. In accordance with these embodiments, treating the donor can lead to reducing graft rejection in a recipient subject as well as reducing the risk of onset of GvHD in the recipient subject, even in cases of mis-matched donor/subject. In some embodiments, a recipient subject can be administered the composition before, during or after, or combination of before, during and after transplantation in order to reduce transplantation rejection and/or GvHD in the subject. Compositions disclosed herein can be administered to the subject or to the donor according to any of the regimens described herein.

In some embodiments, AAT peptides contemplated for use in the compositions and methods described herein are also intended to include any and all of those specific AAT peptides other than the 10 amino acid fragments found in the accompanying sequence listing or AAT peptides of the attached sequence listing depicted supra. Any combination of consecutive amino acids depicting a portion of AAT or AAT-like activity may be used if capable of reducing or eliminating gut complications of GvHD or steroid refractory GvHD in a subject.

In one aspect, the pharmaceutical compositions described herein can be administered orally, systemically, via an implant, intravenously, topically, intrathecally, intratracheally, intracranially, subcutaneously, intravaginally, intraventricularly, intranasally such as inhalation, mixed with grafts by flushing of organ or suspension of cells, or any combination thereof.

In certain embodiments, an advanced-stage GvHD subject has become resistant to the therapeutic effects of steroids and alternative agents are required to treat GvHD and the side-effects associated thereto with an alternative agent. In accordance with these embodiments, any steroid used to reduce graft rejection or inhibit GvHD is contemplated herein where a subject can become resistant to its effects. In certain embodiments, glucocorticoids (steroids), for example methylprednisolone or prednisone given at doses of 0.5-2 mg/kg/day, are one frequently used therapy of acute GVHD. Approximately 40% of subjects receiving this agent achieve satisfactory responses, and steroids can be tapered off without significant flares of GVHD. Responses to these agents depend upon the primary organ involvement and the severity of GVHD manifestations in the subject. Severe involvement of the gastrointestinal tract, for example, has proven challenging to treat, with a fatality rate of around 80% in steroid-non-responsive subjects. Various agents, including anti-thymocyte globulin (ATG), monoclonal antibodies, extracorporeal photopheresis (ECP), and other strategies, have been used to treat steroid-refractory GVHD, but have met with only partial success. In certain embodiments disclosed herein, glucocorticoid refractory subjects having advanced-stage GvHD are contemplated for treatment with AAT regimens. In accordance with these embodiments, these subjects can include those refractory to methylprednisolone. In other embodiments, these subjects can be treated with an initial high dose of AAT (or AAT fusion polypeptide) followed by lower doses of AAT for up to one year after the initial AAT treatment.

Certain embodiments disclosed herein concern methods for inhibiting development of GvHD in a recipient subject scheduled to receive a transplant from an organ, tissue or cell donor including selecting an organ, tissue or cell donor and administering a pharmaceutically acceptable formulation of a composition comprising AAT, a recombinant thereof, a fusion polypeptide thereof, or a RCL mutant thereof or a carboxyterminal fragment thereof to the donor prior to harvesting the organ, tissue or organ from the donor then harvesting the organ, tissue or cells from the organ, tissue or cell donor. In accordance with these embodiments, the organ, tissue or cells can be stored for later use or can be transplanted or implanted in a recipient subject thereby inhibiting or reducing development of transplantation rejection or onset of GvHD in the recipient subject. In other embodiments, a recipient subject can be administered an AAT composition before, during and/or after transplantation.

Any of the embodiments detailed herein may further include one or more a therapeutically effective amount of anti-microbial drugs anti-inflammatory agent, immunomodulatory agent, or immunosuppressive agent or combination thereof.

Non-limiting examples of anti-rejection agents/drugs may include for example cyclosporine, azathioprine, corticosteroids, FK506 (tacrolimus), RS61443, mycophenolate mofetil, rapamycin (sirolimus), mizoribine, 15-deoxyspergualin, and/or leflunomide or any combination thereof.

In addition, other combination compositions of methods disclosed in herein include certain antibody-based therapies. Non-limiting examples include, polyclonal anti-lymphocyte antibodies, monoclonal antibodies directed at the T-cell antigen receptor complex (OKT3, TIOB9), monoclonal antibodies directed at additional cell surface antigens, including interleukin-2 receptor alpha. Antibody-based therapies may be used as induction therapy and/or anti-rejection drugs in combination with the compositions and methods described herein.

In some embodiments, a subject having had bone marrow transplantation demonstrating symptoms or signs of gut-related events of GvHD as recognized in the art can be given a composition of AAT and/or a carboxyterminal derivative of AAT and/or fusion or recombinant polypeptide thereof in order to reduce symptoms or prevent mortality of the subject. In certain aspects, a subject contemplated herein can demonstrate symptoms or sign of gut-related events of GvHD as recognized in the art.

Acute GvHD is a major complication that prevents successful outcomes after allogeneic bone marrow transplantation (BMT), HCT or other hematopoetic cell implantation events, an effective therapy for hematological malignancies and other non-malignant conditions such as leukemia, severe aplastic anemia, lymphoma, multiple myeloma, immune deficiency disorder, solid-tumor cancer, breast, cancer, ovarian cancer among others.

Previous studies demonstrated that donor T cells and host antigen presenting cells along with several proinflammatory cytokines induce GvHD and contribute to its severity. Evidence previously presented demonstrates that AAT can reduce production of pro-inflammatory cytokines, induce anti-inflammatory cytokines and interfere with maturation of dendritic cells. Using well-characterized mouse models of BMT, effects of AAT on GvHD severity have been studied herein. Some embodiments herein concern administration of AAT at late stages after BMT in order to treat steroid-refractory GvHD in a subject.

In certain embodiments, administration of a composition including AAT and/or carboxyterminal derived peptides of AAT reduced gut complications of GvHD compared to a control not receiving the compositions.

In one embodiment, the reduction, prevention or inhibition of rejection of transplantation or side effects thereof associated with one or more of each of the above-recited conditions can be about 10-20%, 30-40%, 50-60%, or more reduction or inhibition due to administration of the disclosed compositions.

In each of the recited methods, an AAT (e.g. mammalian derived) or inhibitor of serine protease activity substance contemplated for use within the methods described herein can include a series of peptides or a peptide including carboxyterminal amino acid peptides corresponding to the carboxyterminus of AAT. Some of these can be pentapeptides. Among this series of peptides, several are equally acceptable in methods disclosed herein including FVFLM (SEQ ID NO: 1), FVFAM (SEQ ID NO: 2), FVALM (SEQ ID NO: 3), FVFLA (SEQ ID NO: 4), FLVFI (SEQ ID NO: 5), FLMII (SEQ ID NO: 6), FLFVL (SEQ ID NO: 7), FLFVV (SEQ ID NO: 8), FLFLI (SEQ ID NO: 9), FLFFI (SEQ ID NO: 10), FLMFI (SEQ ID NO: 11), FMLLI (SEQ ID NO: 12), FIIMI (SEQ ID NO: 13), FLFCI (SEQ ID NO: 14), FLFAV (SEQ. ID) NO. 15), FVYLI (SEQ ID NO: 16), FAFLM (SEQ ID NO: 17), AVFLM (SEQ ID NO: 18), and any combination thereof.

In certain embodiments herein, an AAT peptide or AAT peptides contemplated for use in the compositions and methods of the present disclosure are also intended to include any and all of those specific AAT peptides represented in the Sequence. Any combination of consecutive amino acids simulating AAT or AAT-like activity may be used, such as amino acids 2-12, amino acids 3-14, 4-16, etc.

In each of the above-recited methods, AAT or peptide fragments thereof are contemplated for use in a composition herein for use in certain embodiments as disclosed for the treatment or prevention of symptoms including but not limited to mortalities attributable to late-stage GvHD. In accordance with these embodiments, peptides can include, but are not limited to, amino acid peptides containing 10 consecutive amino acids of AAT such as MPSSVSWGIL (SEQ ID NO: 19); LAGLCCLVPV (SEQ ID NO: 20) SLAEDPQGDA (SEQ ID NO: 21); AQKTDTSHHD (SEQ ID NO: 22) QDHPTFNKIT (SEQ ID NO: 23); PNLAEFAFSL (SEQ ID NO: 24); YRQLAHQSNS (SEQ ID NO: 25); TNIFFSPVSI (SEQ ID NO: 26); ATAFAMLSLG (SEQ ID NO: 27); TKADTHDEIL (SEQ ID NO: 28); EGLNFNLTEI (SEQ ID NO: 29); PEAQIHEGFQ (SEQ. ID) NO. 30); ELLRTLNQPD (SEQ ID NO: 31); SQLQLTTGNG (SEQ ID NO: 32); LFLSEGLKLV (SEQ ID NO: 33); DKFLEDVKKL (SEQ ID NO: 34); YHSEAFTVNF (SEQ ID NO: 35); GDHEEAKKQI (SEQ ID NO: 36); NDYVEKGTQG (SEQ ID NO: 37); KIVDLVKELD (SEQ ID NO: 38); RDTVFALVNY (SEQ. ID NO. 39); IFFKGKWERP (SEQ ID NO: 40); FEVKDTEDED (SEQ ID NO: 41); FHVDQVTTVK (SEQ ID NO: 42); VPMMKRLGMF (SEQ ID NO: 43); NIQHCKKLSS (SEQ ID NO: 44); WVLLMKYLGN (SEQ ID NO: 45); ATAIFFLPDE (SEQ ID NO: 46); GKLQHLENEL (SEQ ID NO: 47); THDIITKFLE (SEQ. ED NO. 48); NEDRRSASLH (SEQ ID NO: 49); LPKLSITGTY (SEQ ID NO: 50); DLKSVLGQLG (SEQ ID NO: 51); ITKVFSNGAD (SEQ ID NO: 52); LSGVTEEAPL (SEQ ID NO: 53); KLSKAVHKAV (SEQ ID NO: 54); LTIDEKGTEA (SEQ ID NO: 55); AGAMFLEAIP (SEQ ID NO: 56); MSIPPEVKFN (SEQ ID NO: 57); KPFVFLMIEQ (SEQ ID NO: 58); NTKSPLFMGK (SEQ ID NO: 59); VVNPTQK (SEQ ID NO: 60), other peptides of 10 amino acids represented in the Sequence Listing, or any combination thereof.

In accordance with embodiments, peptides contemplated herein can be protected or derivitized in by any means known in the art for example, N-terminal acylation, C-terminal amidation, cyclization, etc. In a specific embodiment, the N-terminus of the peptide is acetylated.

### Pharmaceutical Compositions

Embodiments herein provide for administration of compositions to subjects in a biologically compatible form suitable for pharmaceutical administration in vivo. By “biologically compatible form suitable for administration in vivo” is meant a form of the active agent (e.g. pharmaceutical chemical, protein, gene, antibody etc of the embodiments) to be administered in which any toxic effects are outweighed by the therapeutic effects of the active agent. Administration of a therapeutically active amount of the therapeutic compositions is defined as an amount effective, at dosages and for periods of time necessary to achieve the desired result. For example, a therapeutically active amount of a compound may vary according to factors such as the disease state, age, sex, and weight of the individual, and the ability of antibody to elicit a desired response in the individual. Dosage regima may be adjusted to provide the optimum therapeutic response.

In one embodiment, the compound (e.g. chemical, protein, polypeptide etc.) can be administered in a manner known in the art, for example, subcutaneous, intravenous, oral administration, inhalation, transdermal application, intravaginal application, topical application, intranasal or rectal administration. Depending on the route of administration, the active agent may be coated in a material for ease of administration or to protect the compound from the degradation by enzymes, acids and other natural conditions that may inactivate the compound. In certain embodiments, the compound may be orally or intravenously administered. In other embodiments, the compound may be administered intranasally, such as inhalation.

A compound may be administered to a subject in an appropriate carrier or diluent, co-administered with enzyme inhibitors or in an appropriate carrier such as liposomes. The term “pharmaceutically acceptable carrier” or “pharmaceutically acceptable excipient” as used herein is intended to include diluents such as saline and aqueous buffer solutions. It may be necessary to coat the compound with, or co-administer the compound with, a material to prevent its inactivation. The active agent may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use may be administered by any means known in the art. For example, sterile aqueous solutions (where water soluble) or dispersions and sterile powders for the extemporaneous preparation of sterile injectable solutions or dispersion may be used. In all cases, the composition cant be sterile and can be fluid to the extent that easy syringability exists. It might be stable under the conditions of manufacture and storage and may be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of microorganisms can be achieved by heating, exposing the agent to detergent, irradiation or adding various antibacterial or antifungal agents.

Sterile injectable solutions can be prepared by incorporating active compound (e.g. a compound that reduces serine protease activity) in the required amount in an appropriate solvent with one or a combination of ingredients enumerated above, as required, followed by filtered sterilization.

Aqueous compositions can include an effective amount of a therapeutic compound, peptide, epitopic core region, stimulator, inhibitor, and the like, dissolved or dispersed in a pharmaceutically acceptable carrier or aqueous medium. Compounds and biological materials disclosed herein can be purified by means known in the art.

Solutions of the active compounds as free-base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. Prolonged absorption of the injectable compositions can be brought about by the use in the compositions of agents delaying absorption, for example, aluminum monostearate and gelatin.

Upon formulation, solutions will be administered in a manner compatible with the dosage formulation and in such amount as is therapeutically effective. The formulations are easily administered in a variety of dosage forms, such as the type of injectable solutions described above. It is contemplated that slow release capsules, timed-release microparticles, and the like can also be employed. These particular aqueous solutions are especially suitable for intravenous, intramuscular, subcutaneous and intraperitoneal administration.

The active therapeutic agents may be formulated within a mixture to comprise about 0.0001 to 1.0 milligrams, or about 0.001 to 0.1 milligrams, or about 0.1 to 1.0 or even about 1 to 10 gram per dose. Single dose or multiple doses can also be administered on an appropriate schedule for a predetermined condition.

In another embodiment, nasal solutions or sprays, aerosols or inhalants may be used to deliver the compound of interest. Additional formulations that are suitable for other modes of administration include suppositories and pessaries. A rectal pessary or suppository may also be used. In general, for suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

A pharmaceutical composition may be prepared with carriers that protect active ingredients against rapid elimination from the body, such as time-release formulations or coatings. Such carriers include controlled release formulations, such as, but not limited to, microencapsulated delivery systems, and biodegradable, biocompatible polymers, such as ethylene vinyl acetate, polyanhydrides, polyglycolic acid, polyorthoesters, polylactic acid and others are known.

Pharmaceutical compositions are administered in an amount, and with a frequency, as disclosed herein to reduce gut involvement and/or reduce advance-stage GvHD such as Grades III and IV GvHD in a subject in need thereof. Dosage and duration of treatment can be determined by a health professional and administered from 1 to 3 times per day for a predetermined period of time. It will be apparent that, for any particular subject, specific dosage regimens may be adjusted over time according to the individual need. In certain embodiments, doses for administration can be anywhere in a range between about 1.0 mg/kg to about 150 mg/kg. In some embodiments, the dosage range can be between 1.0 mg/kg and 100 mg/kg which can be administered multiple times a day, daily, every other day, biweekly, weekly, monthly etc. In certain embodiments, the range can be about 10.0 mg/kg to about 75 mg/kg introduced to a subject in an initial or follow-on dose. The therapeutically effective amount of AAT, peptides or recombinants thereof can be also measured in molar concentrations and can range between about 1 nM to about 2 mM.

Tablets, troches, pills, capsules and the like containing the active agent can also contain the following agents including, but not limited to, a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent.

### Therapeutic Methods

In one embodiment, methods provide for treating a subject having advanced-stage GvHD with an AAT-containing composition. In accordance with these embodiments, compositions disclosed herein can include a compound capable of inhibiting at least one serine protease or having other activity, for example, AAT or carboxyterminal peptide or fusion polypeptide thereof, or recombinant thereof, or analog thereof.

In certain embodiments, onset of late-stage or advanced-stage GvHD as contemplated herein can be due to transplantation of an organ, tissue or cell, for example, lung, kidney, heart, liver, soft tissue, skin, pancreas, intestine, soft tissue cornea, bone marrow, stem cell, HCT, thymus, pancreatic islet, or other process such as a blood transfusion can also lead to GvHD which can progress to advanced stages.

In another embodiment, methods and compositions described herein can be useful in the therapeutic treatment of graft rejection associated side effects. In a yet another embodiment, late-stage GvHD associated side effects can be reduced or prevented by administration of the compositions as disclosed herein, in order to ameliorate, reduce or eliminate one or more symptoms, side-effects or one or more signs, or prior to onset of one or more severe symptoms or even mortality due to grade 3 or 4 gut involvement. It is contemplated herein that the present compositions and methods can be used to treat a subject requiring chronic therapy.

Desirable blood levels may be maintained by continuous infusion to provide about 0.01-5.0 mg/kg/hr or by intermittent infusions containing about 1.0-150 mg/kg of the active ingredient(s). Buffers, preservatives, antioxidants and the like can be incorporated as required. It is intended herein that the ranges recited also include all those specific percentage amounts between the recited ranges.

### Alpha-1 Antitrypsin

It is to be understood that the present disclosure is not limited to the examples described herein, and other serine proteases known in the art can be used within the limitations described herein if are comparible to AAT. For example, one of skill in the art can easily adopt other serine protease inhibitors known in the art.

AAT is a glycoprotein having two principle forms with a single nucleotide change leading to a single amino acid change. These two principle forms are contemplated of use in methods disclosed herein. These naturally-occurring or native forms of AAT can be used alone, where a carboxyterminal peptide is obtained, a recombinant or as a fusion polypeptide (e.g. AAT-Fc where the Fc is intact or a mutant having truncations in the hinge region or other modification). Human AAT is a single polypeptide chain with no internal disulfide bonds and only a single cysteine residue normally intermolecularly disulfide-linked to either cysteine or glutathione. The reactive site of AAT contains a methionine residue, which is labile to oxidation upon exposure to tobacco smoke or other oxidizing pollutants. Such oxidation reduces the elastase-inhibiting activity of AAT but can maintain other AAT activities of use herein; therefore substitution of another amino acid at that position, e.g., alanine, valine, glycine, phenylalanine, arginine or lysine, produces a form of AAT which is more stable. AAT can be represented by sequences provided herein or any other AAT sequence as known in the art, see for example the attached sequence listing.

It is contemplated that any of the attached sequences, for example, AAT-Fc, a full-length version of AAT linked to an IgG Fc having a hinge or having a hinge modification, a recombinant form of AAT, can be used at a reduced concentration (approximately 10 times less concentrated than naturally-occurring AAT or plasma isolated AAT or commercially available composition of AAT). In certain embodiments, it is contemplated that a subject having advanced-stage GvHD can be treated on day 1 with about 1.0 mg/kg to about 15.0 mg/kg and any follow-on or maintenance treatment can be reduced accordingly or as evaluated by a healthcare professional.

There is an extensive clinical experience using different forms of AAT to treat patients with genetic AAT deficiency. No long-term negative effects have been detected to date.

### Isolated Proteins for Use in the Compositions and Methods Described Herein

One aspect of the present invention pertains to proteins, polypeptides and portions thereof. In one embodiment, the native polypeptide can be isolated from cells or tissue sources by an appropriate purification scheme using standard protein purification techniques. In another embodiment, polypeptides are produced by recombinant DNA techniques. Alternative to recombinant expression, a polypeptide can be synthesized chemically using standard peptide synthesis techniques.

Recombinant unmodified and mutant variants of AAT produced by genetic engineering methods are also known (see for example U.S. Pat. No. 4,711,848). The nucleotide sequence of human AAT and other human AAT variants has been disclosed in international published application No. WO 86/00,337, the entire contents of which are incorporated herein by reference. This nucleotide sequence may be used as starting material to generate all of the AAT amino acid variants and amino acid fragments depicted herein, using recombinant DNA techniques and methods known to those of skill in the art. Fusion polypeptides are also contemplated where for example an immunoglobulin molecule can be fused to AAT or carboxyterminal fragment thereof. In certain embodiments, the immunoglobulin molecule is Fc derived from IgG1, IgG2, IgG3, IgG4 or IgGD.

An isolated and/or purified or partially purified AAT protein or biologically active portion thereof may be used in any embodiment described herein. An AAT protein that is substantially free of cellular material includes preparations of protein having less than about 30%, 20%, 10%, or 5% (by dry weight) of heterologous protein. When the protein or biologically active portion thereof is recombinantly produced, it can also be substantially free of culture medium. When the AAT protein is produced by chemical synthesis, it is preferably substantially free of chemical precursors or other chemicals. Accordingly, such preparations of the protein have less than about 30%, 20%, 10%, and 5% (by dry weight) of chemical precursors or compounds other than the polypeptide of interest.

Biologically active portions of an AAT polypeptide described herein include polypeptides including amino acid sequences sufficiently identical to or derived from the amino acid sequence of the protein (e.g., the amino acid sequence shown in any of the attached sequence listing, which exhibit at least one activity of the corresponding full-length AAT protein). A biologically active portion of a protein can be a polypeptide, which is, for example, 5, 10, 25, 50, 100 or more amino acids in length. Moreover, other biologically active portions, in which other regions of the protein are deleted, can be prepared by recombinant techniques and evaluated for one or more of the functional activities of the native form of a polypeptide described herein.

In some embodiments, polypeptides having the amino acid sequence of those listed in the sequence listing are contemplated among others known in the art. Other useful proteins are substantially identical (e.g., at least about 45%, at least 55%, 65%, 75%, 85%, 95%, or 99%) to any of the listed sequences, and retain the functional activity of the protein of the corresponding naturally-occurring AAT protein yet differ in amino acid sequence due to natural allelic variation or mutagenesis.

Proteins or polypeptides contemplated herein may be administered as free peptides or pharmaceutically acceptable salts thereof. The proteins or polypeptides can be administered to individuals as a pharmaceutical composition, which can include the protein or polypeptide and/or pharmaceutical salts thereof with a pharmaceutically acceptable carrier.

In other embodiments and as disclosed herein, variants of the proteins or polypeptides described herein are contemplated. For example, such variants can have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function.

Variants of a protein of the present disclosure which function as either agonists (mimetics) or as antagonists can be identified by screening combinatorial libraries of mutants, e.g., truncation mutants, of the protein for agonist or antagonist activity.

### Fusion Polypeptides

In other embodiments, proteins or polypeptides having similar activity of AAT can be part of a fusion polypeptide. In one example, a fusion polypeptide may include AAT (e.g. mammalian AAT) or an analog thereof or carboxyterminal peptide derivative thereof and a different amino acid sequence that may be heterologous to the AAT.

In yet other embodiments, a fusion polypeptide contemplated of use in methods of disclosed herein can additionally include an amino acid sequence that is useful for identifying, tracking or purifying the fusion polypeptide, e.g., a FLAG or HIS tag sequence. The fusion polypeptide can include a proteolytic cleavage site that can remove the heterologous amino acid sequence from the compound capable of serine protease inhibition, such as mammalian α1-antitrypsin or analog thereof.

In one embodiment, fusion polypeptides can be produced by recombinant DNA techniques. Alternative to recombinant expression, a fusion polypeptide can be synthesized chemically using standard peptide synthesis techniques. The present disclosure also provides compositions that comprise a fusion polypeptide described herein and a pharmaceutically acceptable carrier, excipient or diluent.

In one embodiment, the fusion protein can include a heterologous sequence that is derived from a member of the immunoglobulin protein family, for example, an immunoglobulin constant region, e.g., a human immunoglobulin constant region such as a human IgG1 or other suitable IgG constant region. The fusion protein can, for example, include a portion of AAT, analog thereof or carboxyterminal fragment thereof fused with the amino-terminus or the carboxyl-terminus of an immunoglobulin constant region, as disclosed, e.g., in U.S. Pat. No. 5,714,147, and U.S. Pat. No. 5,116,964. In accordance with these embodiments, the FcR region of the immunoglobulin may be either wild-type or mutated. In certain embodiments, methods disclosed herein can utilize an immunoglobulin fusion protein that does not interact with an Fc receptor and does not initiate ADCC reactions. In such instances, the immunoglobulin heterologous sequence of the fusion protein can be mutated to inhibit such reactions. See, e.g., U.S. Pat. No. 5,985,279 and WO 98/06248. It is contemplated that IgG1, IgG 2, IgG3 or IgG4 or IgGD or other related immunoglobulin can be used to formulate a fusion polypeptide.

In yet another embodiment, AAT, analog thereof, or carboxyterminal fragment of AAT forming a fusion protein includes a GST fusion protein in which is fused to the C-terminus of GST sequences. Fusion expression vectors and purification and detection means are known in the art.

Expression vectors can routinely be designed for expression of a fusion polypeptide described herein in prokaryotic (e.g., E. coli) or eukaryotic cells (e.g., insect cells (using baculovirus expression vectors), yeast cells or mammalian cells (e.g. CHO cells)) by means known in the art.

Expression of proteins in prokaryotes may be carried out by means known in the art. Such fusion vectors typically serve three purposes: 1) to increase expression of recombinant protein; 2) to increase the solubility of the recombinant protein; and 3) to aid in the purification of the recombinant protein by acting as a ligand in affinity purification.

In yet another embodiment, a nucleic acid is expressed in mammalian cells using a mammalian expression vector as described in the art. In another embodiment, the recombinant mammalian expression vector is capable of directing expression of the nucleic acid preferentially in a particular cell type (e.g., tissue-specific regulatory elements are used to express the nucleic acid) such as pancreas-specific promoters and mammary gland-specific promoters (e.g., milk whey promoter). A host cell can be any prokaryotic (e.g., E. coli) or eukaryotic cell (e.g., insect cells, yeast or mammalian cells). Vector DNA can be introduced into prokaryotic or eukaryotic cells via conventional transformation or transfection techniques.

### Combination Therapies

In each of the aforementioned methods, the use of AAT or analog thereof or fusion polypeptide thereof or carboxyterminal polypeptide of AAT, alone or in combination with standard immunosuppressive agents enables treatment of late-stage GvHD having gut involvement. This combination therapy will expand the eligible subject population able to receive this form of treatment.

In each of the aforementioned aspects and embodiments, combination therapies other than those already enumerated above are also specifically contemplated herein. In particular, the compositions described herein may be admininistered with one or more macrolide or non-macrolide antibiotics, anti-bacterial agents, anti-fungals, anti-viral agents, and anti-parasitic agents.

Examples of anti-bacterial agents include, but are not limited to, penicillins, quinolonses, aminoglycosides, vancomycin, monobactams, cephalosporins, carbacephems, cephamycins, carbapenems, and monobactams and their various salts, acids, bases, and other derivatives.

Anti-fungal agents include, but are not limited to, caspofungin, terbinafine hydrochloride, nystatin, and selenium sulfide.

Anti-viral agents include, but are not limited to, gancyclovir, acyclovir, valacylocir, amantadine hydrochloride, rimantadin and edoxudine

Examples of macrolide antibiotics that may be used in combination with the composition described herein include but are not limited to synthetic, semi-synthetic or naturally occurring macrolidic antibiotic compounds: methymycin, neomethymycin, YC-17, litorin, TMP-SSX, erythromycin A to F, and oleandomycin. Examples of preferred erythromycin and erythromycin-like compounds include: erythromycin, clarithromycin, azithromycin, and troleandomycin.

Anti-parasitic agents include, but are not limited to, pirethrins/piperonyl butoxide, permethrin, iodoquinol, metronidazole, co-trimoxazole (sulfamethoxazole/trimethoprim), and pentamidine isethionate.

In another aspect, in a method described herein, one may, for example, supplement the composition by administration of a therapeutically effective amount of one or more an anti-inflammatory or immunomodulatory drugs or agents. By “anti-inflammatory drugs”, it is meant, e.g., agents which treat inflammatory responses, i.e., a tissue reaction to injury, e.g., agents which treat the immune, vascular, or lymphatic systems.

Anti-inflammatory or immunomodulatory drugs or agents suitable for use include, but are not limited to, interferon derivatives, (e.g., betaseron); prostane derivatives, (e.g., compounds disclosed in PCT/DE93/0013, iloprost, cortisol, dexamethasone; immunsuppressives, (e.g., cyclosporine A, FK-506 (mycophenylate mofetil); lipoxygenase inhibitors, (e.g., zileutone, MK-886, WY-50295); leukotriene antagonists, (e.g., compounds disclosed in DE 40091171 German patent application P 42 42 390.2); and analogs; peptide derivatives, (e.g., ACTH and analogs); soluble TNF-receptors; TNF-antibodies; soluble receptors of interleukins, other cytokines, T-cell-proteins; antibodies against receptors of interleukins, other cytokines, and T-cell-proteins.

### Kits

In certain embodiments, kits for use with the methods described above are provided. Proteins, polypeptides or recombinant or fusion polypeptides may be employed for use in any of the disclosed methods. In addition, other agents such as anti-bacterial agents, immunosuppressive agents, anti-inflammatory agents may be provided in the kit. The kits will thus can include, in suitable container means, a protein or a peptide or analog agent, and optionally one or more additional agents.

The kits may further include a suitably aliquoted composition of the encoded protein or polypeptide antigen, whether labeled or unlabeled, as may be used to prepare a standard curve for a detection assay.

The container means of the kits will generally include at least one vial, test tube, flask, bottle, syringe or other container means, into which the antibody or antigen may be placed, and preferably, suitably aliquoted. Where a second or third binding ligand or additional component is provided, the kit will also generally contain a second, third or other additional container into which this ligand or component may be placed. The kits will also typically include a means for containing the antibody, antigen, and any other reagent containers in close confinement for commercial sale. Such containers may include injection or blow-molded plastic containers into which the desired vials are retained.

### Examples

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

It is contemplated that any of the attached sequences, for example, AAT-Fc, a full-length version of AAT linked to an IgG Fc having a hinge or having a hinge modification, a recombinant form of AAT, can be used at a reduced concentration (approximately 10 times less concentrated than naturally-occurring AAT or plasma isolated AAT or commercially available composition of AAT). In certain embodiments, it is contemplated that a subject having advanced-stage GvHD can be treated on day 1 with about 1.0 mg/kg to about 15.0 mg/kg and any follow-on or maintenance treatment can be reduced accordingly or as evaluated by a healthcare professional.

### Patient and GVHD Characteristics

Exemplary patient and transplant characteristics are presented in exemplary Table 1. Median time to onset of acute GVHD was 28 days after transplantation in these exemplary patients. All patients had overall grades III or IV GVHD. All patients had baseline diarrhea volumes averaging more than one liter/day, and 4 of these had volumes ≥2 liters/day. Patients also underwent upper and lower gastrointestinal endoscopies and biopsies at the initiation of AAT treatment, documenting stage 4 involvement of the colon in all patients studied.

In certain studies, it was demonstrated that a correlation of stool content of AAT (and AAT clearance) with GVHD of the small bowel was observed in both pediatric and in adult patients. The loss of AAT in stool is a reflection of intestinal injury and, presumably, compromises the protective local and systemic effects of AAT. It was speculated that the administration of exogenous AAT could: a) block pro-inflammatory cytokines and b) replace intestinal losses of AAT. Then, the exogenous AAT would promote healing of GVHD-related gut injury, which should also contribute to a reduction in intestinal losses. Furthermore, it was proposed that the shift in cytokine production would lead to an environment that would facilitate the establishment of tolerance.

Mild bilirubin elevations (<3 mg/dL) was present in three patients at the start of AAT therapy. No patients had evidence of skin GVHD. GVHD grading at the start of steroid therapy and at the time of initiating AAT as secondary treatment is illustrated in exemplary Table 2.

TABLE 1
Patient characteristics, diagnoses and transplant regimens.

| | | | | | | | | | |
|---|---|---|---|---|---|---|---|---|---|
| Patient | Number of | Age | | | | HLA | Source of | Conditioning | GVHD |
| ID | Transplant | (years) | Sex | Diagnosis | Donor | Match | Stem Cells | Regimen | Prophylaxis |

Cohort 1

| | | | | | | | | | |
|---|---|---|---|---|---|---|---|---|---|
| 1 | 1 | 36.5 | M | MDS | Not Related | Y | PBSC | CY, H-TBI(1200) | TAC + MTX |
| 2 | 1 | 57.7 | M | AML | Sibling | Y | PBSC | CLOFAR, TBI(200) | CSP + MMF |
| 3 | 1 | 58.3 | F | CLL | Not Related | Y | PBSC | FLU, TBI(200) | CSP + MMF |
| 4 | 2 | 59.3 | M | NHL | Not Related | Y | PBSC | FLU, TBI(200) | CSP + Siro + MMF |
| 5 | 1 | 35.7 | M | ALL | Not Related | N | CORD | CY, LI(400), FLU, H-TBI(1320) | CSP + MMF |
| 6 | 2 | 50.2 | F | ALL | Not Related | N | CORD | CY, TEPA, FLU, F-TBI(400) | CSP + MMF |

Cohort 2

| | | | | | | | | | |
|---|---|---|---|---|---|---|---|---|---|
| 7 | 1 | 49.9 | M | AML | Not Related | Y | PBSC | BU, CY | TAC + MTX |
| 8 | 1 | 73.3 | M | AML | Not Related | Y | PBSC | BC8SA, RDB, FLU, TBI(200) | CSP + MMF |
| 9 | 2 | 26.8 | M | NHL | Sibling | Y | PBSC | FLU, TBI(200) | CSP + MMF |
| 10 | 1 | 29.0 | M | ALL | Not Related | Y | PBSC | CY, H-TBI(1200) | TAC + MTX |
| 11 | 1 | 69.7 | M | MDS | Sibling | Y | PBSC | TREO, FLU | CSP + MMF |
| 12 | 1 | 44.4 | M | AML | Sibling | Y | PBSC | TREO, FLU, TBI(200) | TAC + MMF |

Abbreviations: ALL = acute lymphocytic leukemia; AML = acute myeloid leukemia; BC8SA = streptavidin-conjugated anti-CD45 antibody; BU = busulfan; CLOFAR = clofarabine; CSP = cyclosporine; CY = cyclophosphamide; F = female; FLU = fludarabine; GVHD = graft-versus-host disease; HLA = human leukocyte antigen; H-TBI = hyperfractionated total body irradiation; LI = localized irradiation; M = male; MDS = myelodysplastic syndrome; MMF = mycophenolate mofetil; MTX = methotrexate; NHL = non-Hodgkin lymphoma; PBSC = peripheral blood stem cells; RDB = radio-labeled DOTA-biotin; siro = sirolimus; TAC = tacrolimus; TBI = total body irradiation; TEPA = thiotepa; TREO = treosulfan; Y = yes, N = no. Tx = transplant 1 = first, 2 = second.

### Toxicity and Clinical Outcome

As evaluated in these exemplary methods, there was no clinically apparent toxicity in any patient due to AAT treatment. Patients 8 and 9 of the study discontinued treatment after AAT doses 5 and 7, respectively. The remaining 10 patients received all eight prescribed doses.

Clinical responses to AAT were observed in 8 patients, and 4 responses were complete (patients 3, 4, 7, and 10) by criteria relevant in the art, CIBMTR criteria. Further, additional patients 1, 2, 5, and 6 experienced complete responses in the gastro-intestinal tract (gut involvement of GvHD was eliminated), using the acceptable criteria of the Acute GVHD Activity Index. In certain patients, liver function abnormalities persisted (patients 1, 5, and 6). One of these (patient 1), eventually died with liver failure. A liver biopsy showed adenovirus in addition to histologic evidence of GVHD. Patient 11 had a transient improvement as determined by reduction in stool volume but developed progressive liver disease. Progressive liver disease also developed in one non-responder (patient 8) and one patient with an initial partial response (patient 5). Patients 3, 4, 7 and 10 had complete responses.

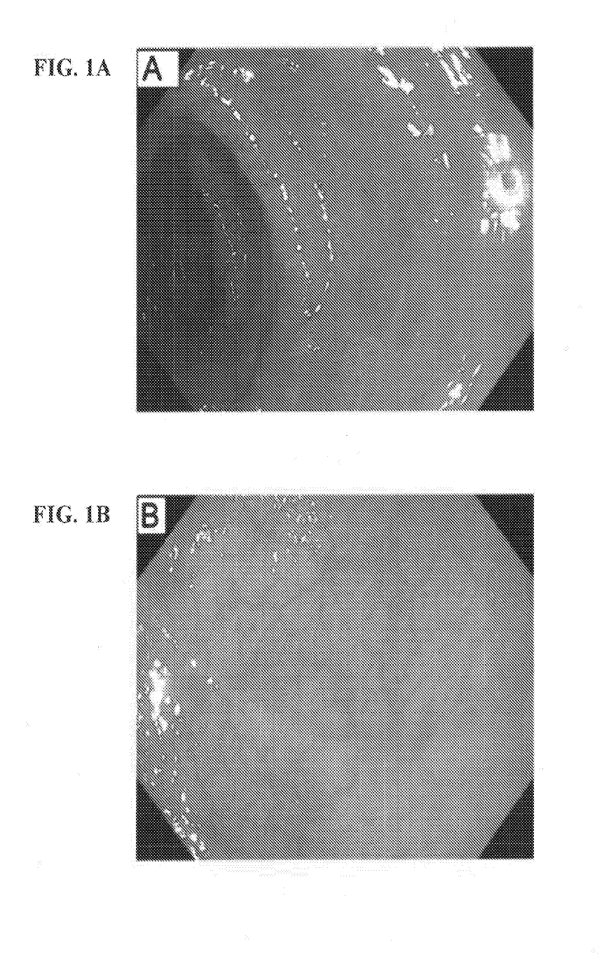

Two of the four patients with complete resolution of the intestinal manifestations required additional systemic GVHD treatment after completing AAT but before study day 28. At study day 28, six of 11 patients who were receiving parenteral nutrition at the start of AAT therapy no longer required parenteral nutritional support, and 8 of 10 evaluable patients had reductions in stool volumes by greater than 50% from baseline. In the 4 patients with complete responses and in 3 of 4 patients with intestinal responses, it was possible to reduce the doses of steroids. Thus, eight patients showed resolution of intestinal GVHD (as defined in Methods, which had been the indication for initiating salvage therapy with AAT), and four of these had complete responses as determined by CIBMTR criteria. Follow-up endoscopies and biopsies at 7-15 days after initiating therapy with AAT documented morphological and histological responses in the gastrointestinal tract are represented. (See for example, FIG. 1).

Tertiary treatment of non-responding patients or patients with incomplete or transient responses varied but included ATG and extracorporeal photopheresis. At the time of this analysis 6 patients were alive >134 to >850 days after transplantation, while 6 had died, 58-208 days after transplantation. Causes of death are illustrated in Table 2. In all 6 patients, GVHD was a contributing factor.

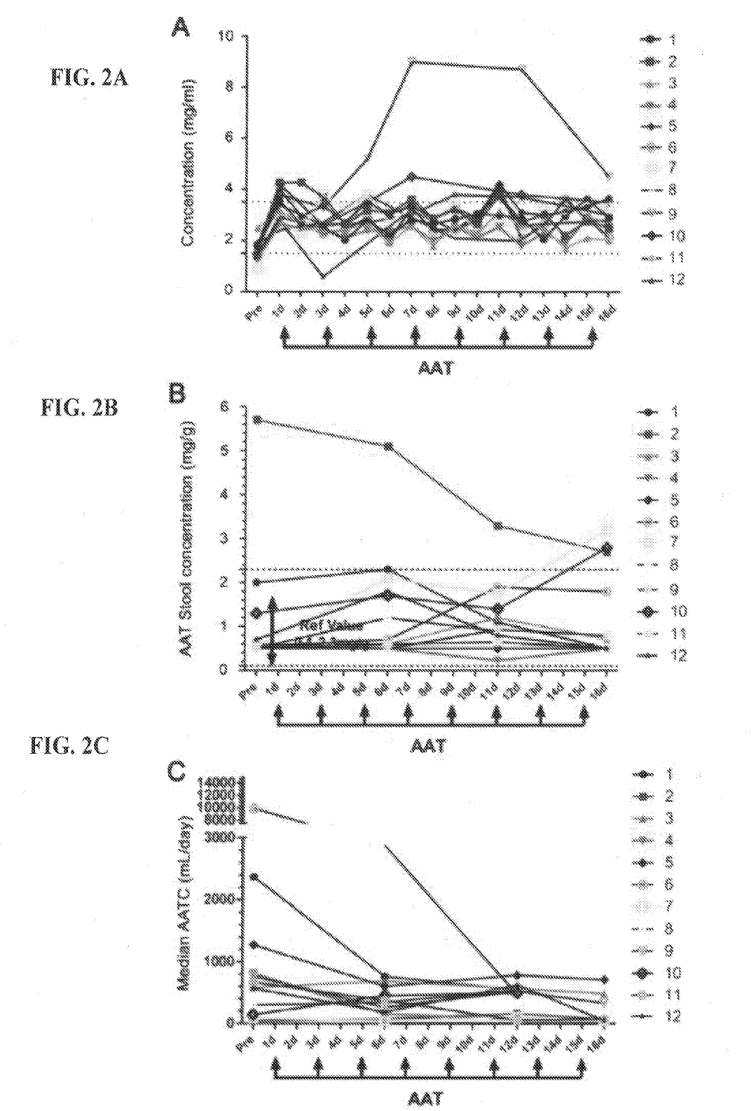

FIG. 2 represents an exemplary illustration of plasma levels of AAT in patients (in comparison to the normal range, ≈1.5-3.5 mg/ml), and stool, and the calculated AAT clearance. All patients showed an increase in plasma levels following the start of AAT treatment (p=0.0005, pre-treatment to day 1). The AAT content of stool decreased (p=0.10, pre-treatment to day 7), and AAT clearance decreased over the course of treatment (p=0.09, pre-treatment to day 7). However, with the number of patients studied, there was no clear correlation between the rate of decline in clearance and clinical response, and changes in clearance were similar in the two cohorts (p=0.26).

TABLE 2
GVHD grade, treatment, and outcome

| | | | AAT Start | | | | | |
|---|---|---|---|---|---|---|---|---|
| Steroid Start | | | | Post-HCT/Post | | | | Days to |
| | Day relative | Response to | GVHD | Steroid Start | Response to | Steroid | Cause of | Death/Date |
| GVHD^{†} | to HCT | steroids | Grade^{‡} | (days)** | AAT | Taper | Death | of Last Contact |

Cohort 1

| | | | | | | | | | |
|---|---|---|---|---|---|---|---|---|---|
| 1 | II (s.g) | 49 | NO | III (g, l) | 60/11 | YES (liver+) | Yes | Liver failure | 116 |
| 2 | II (s.g) | 31 | Transient | IV (g) | 60/29 | YES (incomplete) | Yes | ARDS | 172 |
| 3 | II (g) | 9 | YES, then | III (g) | 569/560 | YES | Yes | N/A | >850 |
| | | | re-exacerbation | | | | | | |
| 4 | II (g) | 33 | NO | IV (g) | 44/11 | YES | Yes | N/A | >537 |
| 5 | II (g) | 59 | Progression, GI | IV (g, l) | 70/11 | YES (liver+) | No | GVHD*, MOF | 208 |
| 6 | II (g) | 22 | Transient | IV (g, l) | 36/14 | YES (liver+) | Yes | Unknown | 58 |

Cohort 2

| | | | | | | | | | |
|---|---|---|---|---|---|---|---|---|---|
| 7 | II (s.g) | 17 | NO | III (g) | 70/53 | YES | Yes | N/A | >307 |
| 8 | III (s.g) | 61 | NO | IV (g) | 69/8 | NO | No | GVHD* | 99 |
| 9 | II (s.g) | 52 | NO | III (g) | 59/7 | NO | No | N/A | >191 |
| 10 | II (g) | 24 | NO | IV (g) | 35/11 | YES | Yes | N/A | >173 |
| 11 | III (g) | 26 | NO | III (g) | 50/24 | Transient (diarrhea | No | GVHD*, MOF | 65 |
| | | | | | | volume reduced) | | | |
| 12 | II (g) | 32 | NO | III (g) | 50/18 | NO | No | N/A | >134 |

**Indicates days on steroids before initiation of AAT therapy
^{†}All patients were “standard” risk category in Minnesota criteria [30] at the start of steroids.
^{‡}All patients had stage 3-4 lower GI tract involvement.
Abbreviations: AAT = alpha-1 antitrypsin (Glassia ®); ARDS = acute respiratory distress syndrome; GI = gastrointestinal; MOF = multiple organ failure; N/A = not applicable; (g) = gut involvement; (s) = skin involvement; (l) = liver involvement

Cytokine Protein and mRNA Level

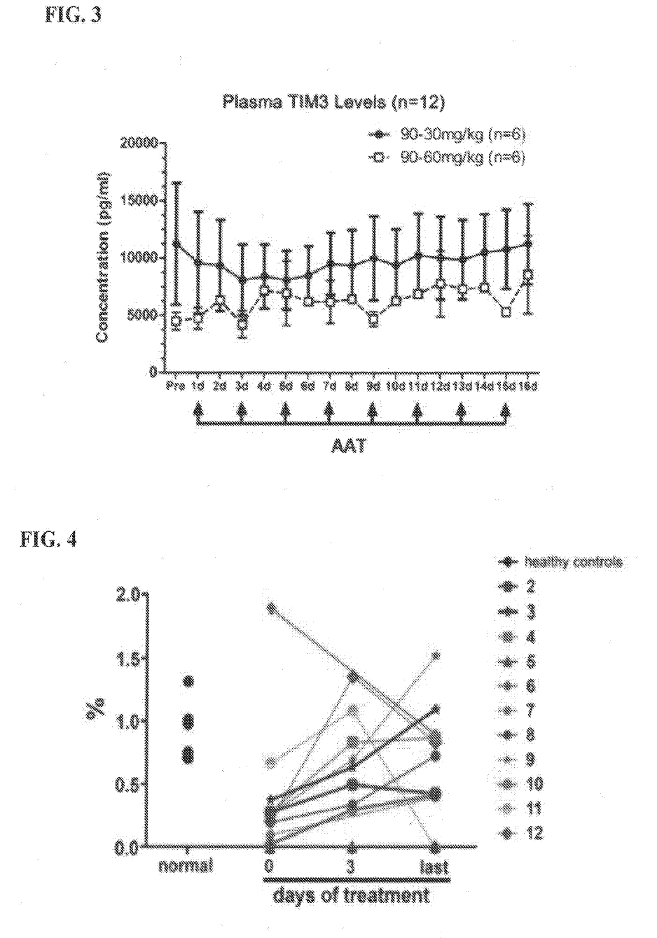

In one exemplary method, cytokine levels at the initiation of treatment did not appear to correlate with GVHD severity, and changes during therapy were inconsistent. Of note, plasma TIM3 levels were lower in patients in cohort 2 (given the higher dose of AAT) but not significantly so (p=0.53), but there was no decline in plasma levels over time (See for example, FIG. 3).

### Effects of AAT on Regulatory T Cells

All patients were lymphocytopenic at the start of AAT (249±306/μL), and during therapy lymphocyte counts declined further, to 80±26/μL at the completion of AAT (day 15 of therapy). However, this difference was not significant (p=0.19), nor was there a significant difference in average lymphocyte levels between the two cohorts (p=0.87). Tregs were analyzed in 11 patients. There was a clear increase in the proportion of CD4+CD25+FoxP3+lymphocytes following initiation of AAT therapy in 10 patients (FIG. 4). However, including the patient with a decline, the increase was not significant overall (p=0.23).

Prior to this study, it was observed that six-month survival was superior in younger patients, and inferior among patients with grade IV GVHD. Using equine ATG as second line therapy 6-month survival was about 44% among 79 patients, median age 27 years, but only 5% of these had grade IV GVHD. Therefore, a 50% survival among patients in the instant study disclosed herein, with a median age of 50 years, all of whom had grades III-IV GVHD with stage 4 intestinal GVHD is significant. These data illustrate that AAT is well tolerated in the treatment of steroid-refractory GVHD having gut involvement. Salvage therapy with AAT was able to induce responses of severe acute GVHD, particularly of the intestinal tract, in patients who had failed to respond to steroids. While observations with secondary therapy cannot be extrapolated directly to up-front treatment, these results together with other published data provide a rational for the use of AAT up-front.

### Patients and Methods

### Study Objectives

In certain exemplary methods, observations included a) determining the safety and tolerability of AAT as salvage therapy in patients with steroid refractory acute GVHD, b) characterizing pharmacokinetic and pharmacodynamic effects of AAT on pro-inflammatory cytokines, and the spectrum of peripheral blood T cells, and c) estimating clinical responses of steroid-refractory acute GVHD having gut involvement to AAT.

### Patients

Patients were enrolled in a study. Eligible were patients who had undergone allogeneic HCT from related or unrelated donors after various conditioning regimens and who had developed acute GVHD that did not show clinical responses to the administration of glucocorticoids, for example, i.v. methylprednisolone at 2 mg/kg/day, given for a minimum of 5 days. All these patients underwent upper and lower gastrointestinal endoscopies to confirm histologic gut involvement by GVHD and determine the extent of GVHD. Patients who had received systemic therapy for acute GVHD other than steroids were excluded, as were patients with manifestations of chronic GVHD or acute/chronic GVHD overlap syndrome, recurrent hematologic malignancy, severe organ dysfunction, or uncontrolled infection.

Patient characteristics (cohorts 1 and 2 of the study, 6 patients per cohort) are presented in exemplary Table 1. Patients were 26.8 to 73.3 (median 50) years old and weighed 64.0-118.9 kg (mean±SD 85.9±19.6 kg). Nine patients had received their first transplant and 3 patients their second transplant. Eight patients were transplanted from unrelated donors, and 4 from HLA-matched siblings. In 10 patients the source of stem cells was peripheral blood (G-CSF mobilized), while two patients received HLA non-identical cord blood cells.

### Exemplary Treatment

In this exemplary study, this was an open-label dose escalation study of AAT, which enrolled six patients at each dose level, subject to predetermined dose escalation, de-escalation, and stopping rules for toxicity. In one group (cohort 1) of patients they were administered AAT (GLASSIA®; Baxalta/Kamada) at 90 mg/kg on day 1, followed by maintenance doses of 30 mg/kg/day on days 3, 5, 7, 9, 11, 13 and 15. Another group (cohort 2), the initial AAT dose was 90 mg/kg on day 1, followed by 7 maintenance doses of 60 mg/kg/day on the same schedule. In these examples, AAT was administered i.v. at a rate of 0.04 ml/kg/minute. In other methods, the GvHD subject can receive at least 5 or more follow-on doses, 10 or more follow on doses etc.

In certain examples, escalation to a higher dose level can be considered if two or fewer patients experienced toxicity. If three or more of six patients met toxicity criteria in cohort 1, but there was evidence of clinical improvement of GVHD, the dose of AAT was to be de-escalated (cohort 0) as follows: 60 mg/kg on day 1, and subsequently 15 mg/kg for each subsequent dose. If toxicity met stopping criteria (toxicity in three or more patients) and there was no evidence of clinical efficacy in cohort 1, the trial was to be closed. If three or more of the six patients experience toxicity at any dose level, a next lower dose was to be the maximum tolerated dose. Dose limiting toxicities and events that would prompt study closure were defined in the protocol.

During protocol treatment administration of GVHD prophylaxis according to the patient's primary transplant protocol continued. For example, steroids, given as initial therapy for GVHD were also continued through completion of the AAT course, but the dose could be tapered at a rate to be determined by the health professional. Antibiotic prophylaxis and other supportive care were given according to guidelines known in the art.

In another example, other AAT commercial formulations also resulted in a favorable response rate (data not shown) where the patient's disease was under control and the life-theatening GvHD was not an issue. These response rates were greater than 50% Assessment of Response and Toxicity

In certain methods, responses to AAT were assessed sequentially, in two ways, with a final assessment on day 28 after initiating therapy with AAT. First, using established criteria, overall responses were measured; second, using criteria derived from an established Acute GVHD Activity Index, responses in the side effects including the gastrointestinal tract were determined.

By the above criteria, a complete response (CR) was defined as a GVHD score of zero in all evaluable organs concerned. A partial response (PR) was defined as improvement in one or more organs without progression into other organs. Improvement was defined as at least a one point reduction in the organ stage. As a control, for a response to be scored as sustained CR or PR, the patient could not have received systemic treatment for acute GVHD other than steroids and AAT prior to study day 28.

In determining responses of gut GVHD, patients were considered to have achieved a CR if they were able to receive sufficient calories by mouth, not requiring parenteral nutrition, and they passed primarily formed stools. A PR response was defined as a decrease in the requirement of parenteral nutrition to about less than 50% of needed calories, or a reduction of stool volume by greater than 50% for patients with baseline volumes more than 500 mL/day, and without ileus.

All patients were scored for response regardless of whether the patient received all 8 doses of AAT therapy (intent to treat analysis). Patients were monitored for adverse events (AE) throughout treatment and until day 28 after initiation of AAT therapy. AE were to be scored for severity using the known criteria (e.g. as defined in the Common Terminology Criteria for Adverse Events, Version 4.0). Specific labs were collected to monitor for the occurrence of coagulopathies and hepatic dysfunction. A Data Safety Monitoring Board provided study oversight.

### Pharmacokinetic and Correlative Studies

### Pharmacokinetics

Blood samples: In certain methods, AAT blood levels were determined at 24, and 48 hours after the infusion of AAT. AAT concentrations were determined by enzyme-linked immunosorbent assay (ELISA) using 96-well polystyrene plates coated overnight at 4° C. with 0.5 μg/mL mouse anti-human AAT (R&D Systems, Minneapolis, Minn.) in 50 mM Na carbonate, pH 9.5. Other time course evaluations for AAT blood concentrations are contemplated.

Stool samples: In certain methods, stool content of AAT was determined before initiation of treatment and on days 3, 7, 11, and 15. For each collection time point, stool volume was determined over a 24-hour interval. AAT concentrations were measured by standard nephelometric technique. AAT clearance (C) was calculated as follows: C=F×W/p, where F=fecal AAT concentration (mg/gm), W=daily stool weight, and p=serum AAT concentration (mg/dL). A clearance of ≥27 mL/day suggests the diagnosis of Protein Losing Enteropathy.

### Biomarkers

In other methods, blood samples were collected for the determination of various components, for example, interleukin (IL)-1β, IL-1 receptor antagonist (IL-1Ra), IL-32, IL-10, IL-6, IL-15, TIM3, and ST2 were evaluated before and then sequentially after initiation of treatment. Cytokine protein levels were determined. All patient samples, standards, and controls were analyzed in triplicates. Inter-assay and intra-assay coefficients of variability (CV) were determined to be <10% with an assay sensitivity of <2 pg/mL. Expression of cytokines and other markers at the mRNA level was determined by standard techniques as described. In addition, regulatory T-cells, defined as CD4+CD25+CD127+FoxP3+lymphocytes, were determined by flow cytometry.

### Statistical Analysis

Comparisons between AAT dose groups were analyzed by for example, Wilcoxon rank-sum test, were determined. Paired comparisons of changes over time were analyzed by Wilcoxon signed-rank. Levels of TIM3 and AAT clearance were log-transformed prior to analysis. Average levels across time of plasma TIM3, plasma AAT, and lymphocyte counts were used for comparison between AAT dose groups.

All of the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for treating graft versus host disease (GvHD) in a subject, the method comprising
administering to a subject having steroid-refractory GvHD of grade III or grade IV over a 1 day to 60-day administration period a pharmaceutically acceptable composition comprising a pharmaceutically acceptable excipient and an agent selected from the group consisting of alpha-1 antitrypsin (AAT), a recombinant version thereof, a RCL mutant thereof, and a carboxy-terminal fragment thereof;
wherein the subject is administered a higher dose of the agent on day 1 of the administration period than on one or more of the follow-on administrations thereby treating GvHD in the subject.

2. The method of claim 1, wherein the subject further has at least one characteristic selected from the group consisting of: (a) displays gastrointestinal (GI) tract involvement; (b) displays resistance to effects of glucocorticoids; (c) has had hematopoietic cell transplantation (HCT) transplantation.

3. The method of claim 2, wherein the GI tract involvement of the subject comprises stage 3 or 4.

4. (canceled)

5. The method of claim 2, further comprising evaluating the GI tract involvement in the subject using at least one of upper and lower gastro-intestinal evaluations prior to, during, or after agent treatment.

6. The method of claim 1, wherein at least one applies: (a) administration occurs over a 5: day to 30-day period; (b) administration occurs over a 9-day to 20-day period; (c) the one or more follow-on administrations take place every day or every other day following the day 1 or initial administration.

7-8. (canceled)

9. The method of claim 1, wherein at least one applies: (a) the dose of the agent administered to the subject on day 1 ranges from about 1.0 mg/kg to 150 mg/kg; (b) the dose of the agent administered in at least one follow-on administration ranges from about 1.0 mg/kg to 100 mg/kg; (c) the dose of the agent administered to the subject on day 1 is about 90 mg/kg and the dose of the agent administered to the subject in at least one follow-on administration is about 30 mg/kg to about 60 mg/kg.

10-11. (canceled)

12. The method of claim 1, further comprising administering steroids to the subject during the administration of the agent, wherein the steroid dose administered to the subject is reduced or ceased during or after completion of the agent treatment.

13. The method of claim 1, wherein the composition is administered by at least one route selected from inhalation, subcutaneous, oral, intramuscular, and intravenous.

14-15. (canceled)

16. A method for treating graft versus host disease (GvHD) in a subject the method comprising
administering to a subject having steroid-refractory GvHD of grade III or grade IV GvHD with stage 4 gastrointestinal involvement a pharmaceutically acceptable composition comprising an agent selected from the group consisting of alpha-1 antitrypsin (AAT) and a full-length AAT fusion polypeptide over a 1 to 60-day administration period,
wherein the subject is administered a higher dose of the agent on day 1 of the administration period than on one or more of the follow-on administrations thereby treating GvHD in the subject.

17. The method of claim 16, further comprising administering to the subject one or more selected from the group consisting of an anti-inflammatory agent, an immunosuppressive agent, an immunomodulatory agent, an anti-microbial agent, and any combinations thereof.

18. The method of claim 16, wherein the subject is administered the composition after hematopoietic cell transplantation (HCT) transplantation.

19. The method of claim 16, wherein at least one applies: (a) the subject is administered a single dose of the agent on day 1 and additional doses of the agent every other day for up to 60 days after the day 1 administration; (b) the dose of the agent administered to the subject on day 1 ranges from about 1.0 mg/kg to 150 mg/kg; (c) the dose of agent administered to the subject on at least one follow-on administration ranges from about 1.0 mg/kg to 100 mg/kg; (d) the dose of the agent administered to the subject on day 1 is about 90 mg/kg and the dose of agent administered to the subject on at least one follow-on administration ranges from about 30 mg/kg to 60 mg/kg; (e) the agent is administered to the subject at a rate of about 0.04 ml/kg/minute.

20-23. (canceled)

24. A method for inhibiting development of GvHD in a recipient subject scheduled to receive a transplant from an organ, tissue or cell donor subject, the method comprising:
administering to an organ, tissue, or cell donor subject, prior to harvesting the organ, tissue, or cell from the donor subject, a pharmaceutically acceptable composition comprising an agent selected from the group consisting of alpha-1 antitrypsin (AAT), a recombinant version thereof, a fusion polypeptide thereof, a RCL mutant thereof, and a carboxyterminal fragment thereof;
harvesting the organ, tissue, or cell from the donor subject; and
transplanting or implanting the organ, tissue, or cell from the donor subject treated with the agent into the recipient subject, whereby graft rejection or development of GvHD is inhibited in the recipient subject.

25. The method of claim 24, wherein the recipient subject is administered the composition before transplantation, after transplantation, or both before and after transplantation.

26. The method of claim 24 wherein the donor or recipient subject is human.

27. The method of claim 24, wherein the recipient subject received a mis-matched organ, tissue, or cell implant or transplant.

28. The method of claim 27, wherein the mismatched implant comprises an HCT implantation into the subject.
